(19) United States
(12) Patent Application Publication
(10) Pub. No.: US 2021/0259773 A1
(43) Pub. Date: Aug. 26, 2021

(54) ACCURATE MODELLING OF SCAR FORMATION FOR CARDIAC ABLATION PLANNING

(71) Applicant: Siemens Healthcare GmbH, Erlangen (DE)

(72) Inventors: Chloé Audigier, Bern (CH); Tiziano Passerini, Plainsboro, NJ (US); Eric Lluch Alvarez, Nurnberg (DE); Viorel Mihalef, North Brunswick, NJ (US); Tommaso Mansi, Plainsboro, NJ (US)

(21) Appl. No.: 17/143,623

(22) Filed: Jan. 7, 2021

Related U.S. Application Data
(60) Provisional application No. 62/980,473, filed on Feb. 24, 2020.

(51) Int. Cl. A61B 34/10; G16H 50/20

(52) U.S. Cl. CPC A61B 34/10; A61B 2034/104; A61B 2034/105; G16H 50/20

(57) ABSTRACT

Systems and methods for performing a simulation for an anatomical object of interest are provided. A physiological model of an anatomical object of interest of a patient is generated. Electroanatomical mapping data of the anatomical object of interest is received. The physiological model is updated based on the electroanatomical mapping data of the anatomical object of interest. A simulation for the anatomical object of interest is performed using the updated physiological model. Results of the simulation are output.

### CROSS-REFERENCE TO RELATED APPLICATIONS

This application claims the benefit of U.S. Provisional Application No. 62/980,473, filed Feb. 24, 2020, the disclosure of which is incorporated herein by reference in its entirety.

### TECHNICAL FIELD

The present invention relates generally to cardiac ablation planning, and in particular to the accurate modelling of scar formation for cardiac ablation planning.

### BACKGROUND

Atrial fibrillation is the abnormal rhythm of the heart of a patient, typically characterized by a rapid and irregular heart rate. Atrial fibrillation is associated with increased risk in other heart diseases, such as stroke, heart failure, etc. To treat atrial fibrillation, cardiac ablation may be performed to scar or destroy the cardiac tissue causing the atrial fibrillation. Cardiac ablation is a complex, minimally invasive procedure requiring careful planning and guidance.

Typically, planning and guidance for cardiac ablation of a patient is performed by manually defining ablation lines based on medical imaging of the patient. However, such manual definition of ablation lines is not patient specific, may interrupt the clinical workflow, and does not take into account intra-operative data of the response of the patient during the cardiac ablation procedure.

Recently, computational models have been developed to simulate the heart during atrial fibrillation. Such computational models may be utilized to augment pre-procedural planning by simulating outcomes of cardiac ablation procedures. However, conventional computational models rely only on preoperative data, thereby limiting the fidelity of such conventional computational models. Additionally, conventional computational models have long computation times, prohibiting their use in clinical settings.

### BRIEF SUMMARY OF THE INVENTION

In accordance with one or more embodiments, systems and methods for modelling a heart of a patient are provided. A physiological model of the heart is generated and the physiological model is updated with intraoperative electroanatomical mapping data of the heart. The updated physiological model of the heart may be used to simulate ablation procedures performed on the heart with improved accuracy and fidelity as compared to conventional computational models.

In accordance with one embodiment, systems and methods for performing a simulation for an anatomical object of interest are provided. A physiological model of an anatomical object of interest of a patient is generated. Electroanatomical mapping data of the anatomical object of interest is received. The physiological model is updated based on the electroanatomical mapping data of the anatomical object of interest. A simulation for the anatomical object of interest is performed using the updated physiological model. Results of the simulation are output.

In one embodiment, the electroanatomical mapping data comprises a 3D map of the anatomical object of interest having electrophysiological data represented at anatomical locations thereon. The electrophysiological data may comprise at least one of activation time and potentials, voltage, and propagation.

In one embodiment, the physiological model is updated by refining conduction velocity values based on activation potential values in the electroanatomical mapping data.

In one embodiment, the electroanatomical mapping data comprises intraoperative data received during a medical procedure and a simulation of the medical procedure for the anatomical object of interest is performed using the updated physiological model. In one embodiment, the simulation is a simulation of an ablation procedure on a heart of the patient.

In one embodiment, an optimal therapy is predicted using a trained machine learning based model based on information from the updated physiological model and the results of the simulation. In one embodiment, the physiological model is updated based on intraoperative results of an ablation performed during an ablation procedure and a simulation of a next ablation of the ablation procedure is performed based on the updated physiological model.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a simulation for an anatomical object of interest, in accordance with one or more embodiments;

FIG. 2 shows an exemplary artificial neural network that may be used to implement one or more embodiments;

FIG. 3 shows a convolutional neural network that may be used to implement one or more embodiments; and

FIG. 4 shows a high-level block diagram of a computer that may be used to implement one or more embodiments.

### DETAILED DESCRIPTION

The present invention generally relates to accurate modelling of scar formation for cardiac ablation planning. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Embodiments described herein provide for a physiological model of the heart of a patient for planning a cardiac ablation procedure for the patient. The physiological model is updated with intraoperative electroanatomical mapping data of the patient to more accurately represent the current state of the patient at the time of the cardiac ablation procedure. Advantageously, the physiological model updated in accordance with embodiments described herein may be used by a user (e.g., a clinician) to simulate the ablation procedure and directly observe the predicted output of the ablation procedure for the patient.

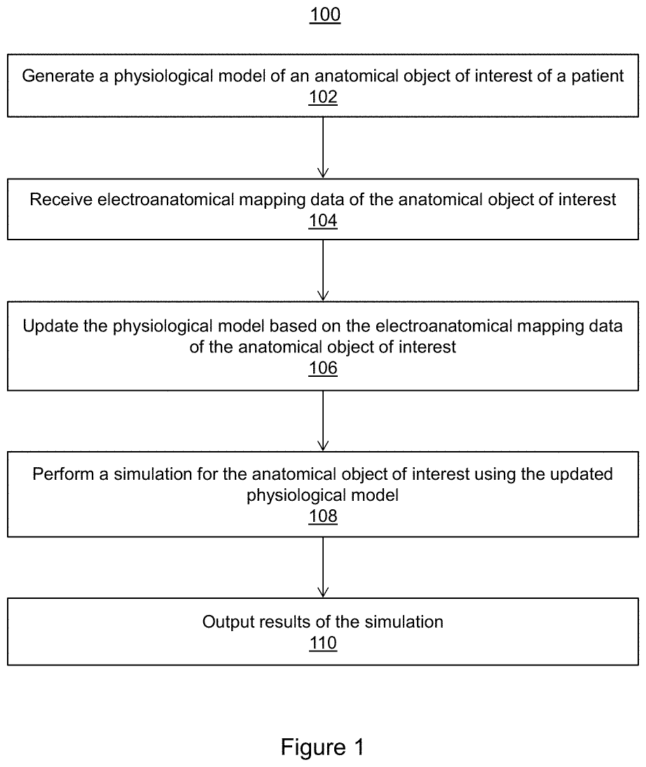

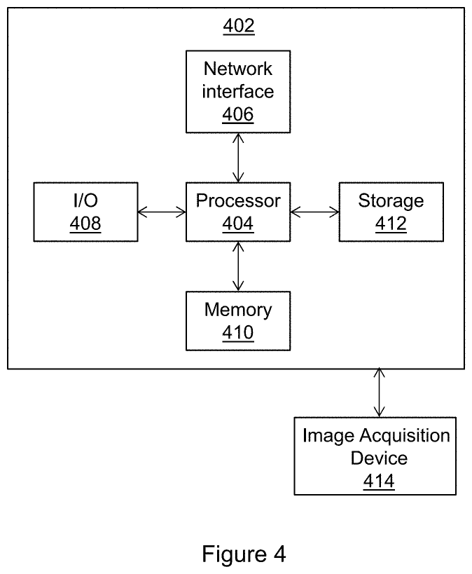

FIG. 1 shows a method 100 for performing a simulation for an anatomical object of interest, in accordance with one or more embodiments. The steps of method 100 may be performed by one or more suitable computing devices, such as, e.g., computer 402 of FIG. 4.

At step 102, a physiological model of an anatomical object of interest of a patient is generated. In one embodiment, the anatomical object of interest is the heart of the patient. However, it should be understood that the anatomical object of interest may be any other suitable anatomical object or objects of interest, such as, e.g., the lungs, liver, brain, musculoskeletal structures, and/or other organs of the patient.

The physiological model may comprise one or more computational models for modelling one or more physiological functions of the anatomical object of interest. For example, the physiological model may comprise a computational model of the heart for modelling movement/mechanics, electrical signal propagation (electrophysiology), blood flow (hemodynamics), and/or disease progression of the heart of the patient. The physiological model may be generated according to any suitable approach. In one embodiment, the physiological model may be generated based on pre-operative data of the patient. In particular, a patient-specific anatomical model of the anatomical object of interest is extracted from medical image data of the patient. The medical image data may comprise 2D (two dimensional) images or 3D (three dimensional) volumes of any suitable modality, such as, e.g., CT (computed tomography), MRI (magnetic resonance imaging), ultrasound, X-ray, DynaCT, PET (positron emission tomography), or any other suitable modality or combination of modalities. The patient-specific anatomical model is then personalized with patient specific physiological parameters determined based on the medical image data and possibly other patient data (e.g., physiological measurements of the patient) to generate the physiological model.

In one embodiment, the physiological model includes a model of heat distribution and scar formation for an ablation procedure performed on the anatomical object of interest. The model of heat distribution and scar formation is based on the bioheat equation for modelling ablation from RF (radiofrequency) currents in RFA (radiofrequency ablation), as well as other types of ablation, such as, e.g., laser ablation, HIFU (high intensity focused ultrasound) ablation, microwave ablation, cryoablation, etc.

At step 104, electroanatomical mapping data of the anatomical object of interest is received. The electroanatomical mapping data may be intraoperatively acquired during a medical procedure (e.g., cardiac ablation) of the patient. In one embodiment, the electroanatomical mapping data is acquired by electroanatomical mapping, which is a catheter-based technique that offers online localization and quantitative assessment of the viability of the anatomical object of interest (e.g., myocardial) by allowing the simultaneous measurement of electrical and mechanical parameters of the anatomical object of interest. For example, the electroanatomical mapping data includes the electrical potential (e.g., in millivolts) value at one or more anatomical points that the catheter was able to capture. The electroanatomical mapping data may be any data mapping electrophysiological data of the patient to anatomical locations on the anatomical object of interest. In one embodiment, the electroanatomical mapping data is a 3D map of the anatomical object of interest having electrophysiological data represented at anatomical locations thereon. Examples of electrophysiological data represented in the electroanatomical mapping data may include local activation time and potentials (e.g., for a cardiac chamber or chambers in relation to a timing of a reference electrogram), unipolar or bipolar voltage, fractionation electrograms, and propagation (e.g., showing spread of activation wave front throughout a cardiac cycle, or showing direction vectors of the wave propagation). In one embodiment, the electroanatomical mapping data is part of the CARTO® data acquired using a Biosense Webster CARTO system, which uses multiple electrodes to acquire the potential (e.g., in millivolts) at several locations of the anatomical object of interest.

At step 106, the physiological model is updated based on the electroanatomical mapping data of the anatomical object of interest. The physiological model may be updated based on the electroanatomical mapping data in substantially real time. In one embodiment, the physiological model is updated by refining conduction velocity values based on activation potential values provided by the electroanatomical mapping data. By updating the physiological model based on the electroanatomical mapping data, the updated physiological model more accurately represents the pathophysiology of the patient. In one embodiment, the update of the physiological model is done using an optimization approach that searches for the combination of physiological parameters (e.g., local conduction velocity) that minimizes the error between the simulated activation times and the measured activation times.

At step 108, a simulation for the anatomical object of interest is performed using the updated physiological model. In one embodiment, the simulation is of a medical procedure, such as, e.g., an ablation procedure performed on the heart of the patient to simulate heat distribution and scar formation. The simulation of the ablation procedure enables a user (e.g., a clinician) to directly observe the prediction of the outcome (e.g., heat distribution, scar formation) of an ablation for effective planning of cardiac ablations.

At step 110, results of the simulation are output. The results of the simulation may be, for example, a predicted outcome of the simulated medical procedure. For example, the results of the simulation can be output by displaying the results of the simulation on a display device of a computer system, storing the results of the simulation on a memory or storage of a computer system, or by transmitting the results of the simulation to a remote computer system. In one embodiment, a plurality of simulations are performed (at step 108) using the updated physiological model and results of the plurality of simulations are presented to the user (at step 110), enabling the user to select the best course of action based on the results of the plurality of simulations.

In one embodiment, a machine learning based model may be trained with deep reinforcement learning to predict an optimal therapy (e.g., an optimal ablation strategy). The machine learning based model may be trained using training data acquired from prior interventions and corresponding patient data. The machine learning based model predicts the optimal therapy based on information from the updated physiological model and simulations (e.g., of a number of realistic ablation procedures) performed using the updated physiological model. The machine learning based model therefore predicts the optimal therapy in a computationally fast manner as compared to conventional approaches, and is therefore suitable for use in clinical settings.

In one embodiment, the physiological model is additionally or alternatively updated based on results of prior medical procedures and/or intraoperative results of the medical procedure. For example, the physiological model may be updated based on intraoperative results of an ablation during an ablation procedure. The physiological model updated in accordance with this embodiment may be utilized to perform the simulation (at step 108) of a next ablation of the ablation procedure. Therefore, the updated physiological model may provide iterative guidance by updating an optimal ablation strategy during the ablation procedure to better treat the patient.

Advantageously, by updating the physiological model based on the electroanatomical mapping data, accuracy and fidelity of the updated physiological model is improved as compared to convention models. As the updated physiological model becomes more accurate, it can be utilized by clinicians with more confidence and therefore help reduce intervention time. The updated physiological model improves cardiac ablation procedures with automatic and fast patient-specific planning.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, embodiments described herein are described with respect to methods and systems for utilizing a trained machine learning based model, as well as with respect to methods and systems for training machine learning based models. Features, advantages or alternative embodiments herein can be assigned to the other claimed objects and vice versa. In other words, claims for methods and systems for training a machine learning based model can be improved with features described or claimed in context of the methods and systems for utilizing a trained machine learning based generator network, and vice versa.

In particular, the trained machine learning based models utilized herein can be adapted by the methods and systems for training the machine learning based models. Furthermore, the input data of the trained machine learning based models can comprise advantageous features and embodiments of the training input data, and vice versa. Furthermore, the output data of the trained machine learning based models can comprise advantageous features and embodiments of the output training data, and vice versa.

In general, a trained machine learning based model mimics cognitive functions that humans associate with other human minds. In particular, by training based on training data, the trained machine learning based model is able to adapt to new circumstances and to detect and extrapolate patterns.

In general, parameters of the machine learning based models can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based models can be adapted iteratively by several steps of training.

In particular, a trained machine learning based model can comprise a neural network, a support vector machine, a decision tree, and/or a Bayesian network, and/or the trained machine learning based model can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

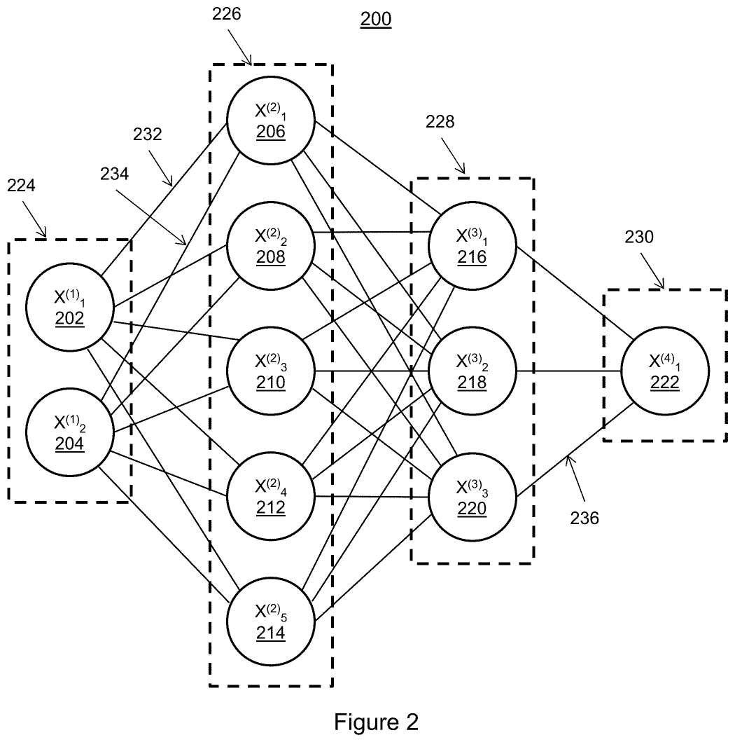

FIG. 2 shows an embodiment of an artificial neural network 200, in accordance with one or more embodiments. Alternative terms for “artificial neural network” are “neural network”, “artificial neural net” or “neural net”. Machine learning networks described herein may be implemented using artificial neural network 200.

The artificial neural network 200 comprises nodes 202-222 and edges 232, 234, . . . , 236, wherein each edge 232, 234, . . . , 236 is a directed connection from a first node 202-222 to a second node 202-222. In general, the first node 202-222 and the second node 202-222 are different nodes 202-222, it is also possible that the first node 202-222 and the second node 202-222 are identical. For example, in FIG. 2, the edge 232 is a directed connection from the node 202 to the node 206, and the edge 234 is a directed connection from the node 204 to the node 206. An edge 232, 234, . . . , 236 from a first node 202-222 to a second node 202-222 is also denoted as “ingoing edge” for the second node 202-222 and as “outgoing edge” for the first node 202-222.

In this embodiment, the nodes 202-222 of the artificial neural network 200 can be arranged in layers 224-230, wherein the layers can comprise an intrinsic order introduced by the edges 232, 234, . . . , 236 between the nodes 202-222. In particular, edges 232, 234, . . . , 236 can exist only between neighboring layers of nodes. In the embodiment shown in FIG. 2, there is an input layer 224 comprising only nodes 202 and 204 without an incoming edge, an output layer 230 comprising only node 222 without outgoing edges, and hidden layers 226, 228 in-between the input layer 224 and the output layer 230. In general, the number of hidden layers 226, 228 can be chosen arbitrarily. The number of nodes 202 and 204 within the input layer 224 usually relates to the number of input values of the neural network 200, and the number of nodes 222 within the output layer 230 usually relates to the number of output values of the neural network 200.

In particular, a (real) number can be assigned as a value to every node 202-222 of the neural network 200. Here, x⁽ⁿ⁾ᵢ denotes the value of the i-th node 202-222 of the n-th layer 224-230. The values of the nodes 202-222 of the input layer 224 are equivalent to the input values of the neural network 200, the value of the node 222 of the output layer 230 is equivalent to the output value of the neural network 200. Furthermore, each edge 232, 234, . . . , 236 can comprise a weight being a real number, in particular, the weight is a real number within the interval [−1, 1] or within the interval [0, 1]. Here, w^{(m,n)}_{i,j} denotes the weight of the edge between the i-th node 202-222 of the m-th layer 224-230 and the j-th node 202-222 of the n-th layer 224-230. Furthermore, the abbreviation w⁽ⁿ⁾_{i,j} is defined for the weight w^{(n, n+1)}_{i,j}.

In particular, to calculate the output values of the neural network 200, the input values are propagated through the neural network. In particular, the values of the nodes 202-222 of the (n+1)-th layer 224-230 can be calculated based on the values of the nodes 202-222 of the n-th layer 224-230 by

x⁽ⁿ⁺¹⁾ⱼ=f(Σᵢxᵢ^{(n)·}w_{i,j}⁽ⁿ⁾.

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions The transfer function is mainly used for normalization purposes.

In particular, the values are propagated layer-wise through the neural network, wherein values of the input layer 224 are given by the input of the neural network 200, wherein values of the first hidden layer 226 can be calculated based on the values of the input layer 224 of the neural network, wherein values of the second hidden layer 228 can be calculated based in the values of the first hidden layer 226, etc.

In order to set the values w^{(m,n)}_{i,j} for the edges, the neural network 200 has to be trained using training data. In particular, training data comprises training input data and training output data (denoted as tᵢ). For a training step, the neural network 200 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 200 (backpropagation algorithm). In particular, the weights are changed according to

w′_{i,j}⁽ⁿ⁾=w_{i,j}⁽ⁿ⁾−γ·δⱼ⁽ⁿ⁾·xᵢ⁽ⁿ⁾

wherein γ is a learning rate, and the numbers δ⁽ⁿ⁾ⱼ can be recursively calculated as

δⱼ⁽ⁿ⁾=(Σₖδₖ⁽ⁿ⁺¹⁾·w_{j,k}⁽ⁿ⁺¹⁾)·f′(Σᵢxᵢ⁽ⁿ⁾·w_{i,j}⁽ⁿ⁾)

based on δ⁽ⁿ⁺¹⁾ⱼ, if the (n+1)-th layer is not the output layer, and

δⱼ⁽ⁿ⁾=(xₖ⁽ⁿ⁺¹⁾−tⱼ⁽ⁿ⁺¹⁾)·f′(Σᵢxᵢ⁽ⁿ⁾·w_{i,j}⁽ⁿ⁾)

if the (n+1)-th layer is the output layer 230, wherein f′ is the first derivative of the activation function, and y⁽ⁿ⁺¹⁾ⱼ is the comparison training value for the j-th node of the output layer 230.

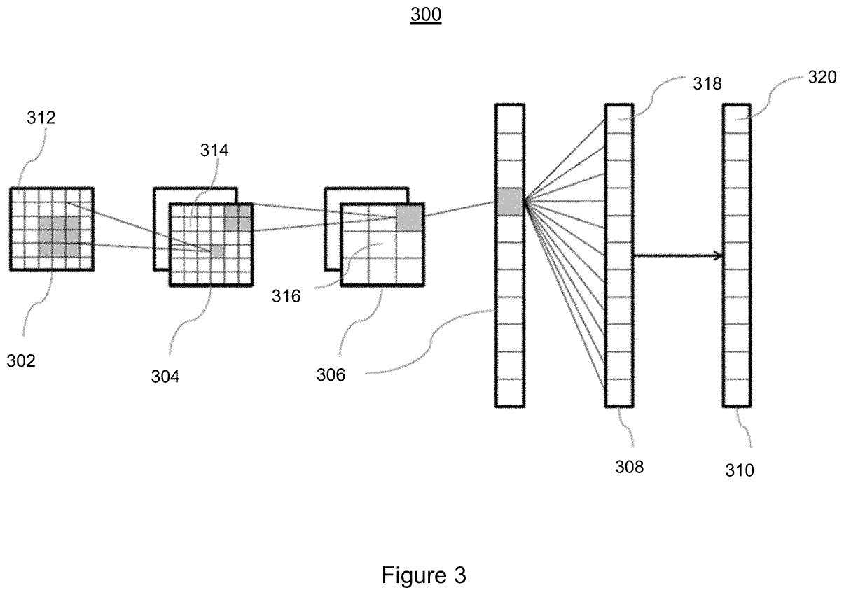

FIG. 3 shows a convolutional neural network 300, in accordance with one or more embodiments. Machine learning networks described herein may be implemented using convolutional neural network 300.

In the embodiment shown in FIG. 3, the convolutional neural network comprises 300 an input layer 302, a convolutional layer 304, a pooling layer 306, a fully connected layer 308, and an output layer 310. Alternatively, the convolutional neural network 300 can comprise several convolutional layers 304, several pooling layers 306, and several fully connected layers 308, as well as other types of layers. The order of the layers can be chosen arbitrarily, usually fully connected layers 308 are used as the last layers before the output layer 310.

In particular, within a convolutional neural network 300, the nodes 312-320 of one layer 302-310 can be considered to be arranged as a d-dimensional matrix or as a d-dimensional image. In particular, in the two-dimensional case the value of the node 312-320 indexed with i and j in the n-th layer 302-310 can be denoted as x⁽ⁿ⁾_{[i,j]}. However, the arrangement of the nodes 312-320 of one layer 302-310 does not have an effect on the calculations executed within the convolutional neural network 300 as such, since these are given solely by the structure and the weights of the edges.

In particular, a convolutional layer 304 is characterized by the structure and the weights of the incoming edges forming a convolution operation based on a certain number of kernels. In particular, the structure and the weights of the incoming edges are chosen such that the values x⁽ⁿ⁾ₖ of the nodes 314 of the convolutional layer 304 are calculated as a convolution x⁽ⁿ⁾ₖ=Kₖ*x⁽ⁿ⁻¹⁾ based on the values x⁽ⁿ⁻¹⁾ of the nodes 312 of the preceding layer 302, where the convolution * is defined in the two-dimensional case as

xₖ⁽ⁿ⁾[i,j](Kₖ*x⁽ⁿ⁻¹⁾)[i,j]=Σ_{i′}Σ_{j′}Kₖ[i′,j′]·x⁽ⁿ⁻¹⁾[i−i′,j−j′].

Here the k-th kernel Kₖ is a d-dimensional matrix (in this embodiment a two-dimensional matrix), which is usually small compared to the number of nodes 312-318 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 312-320 in the respective layer 302-310. In particular, for a convolutional layer 304, the number of nodes 314 in the convolutional layer is equivalent to the number of nodes 312 in the preceding layer 302 multiplied with the number of kernels.

If the nodes 312 of the preceding layer 302 are arranged as a d-dimensional matrix, using a plurality of kernels can be interpreted as adding a further dimension (denoted as “depth” dimension), so that the nodes 314 of the convolutional layer 304 are arranged as a (d+1)-dimensional matrix. If the nodes 312 of the preceding layer 302 are already arranged as a (d+1)-dimensional matrix comprising a depth dimension, using a plurality of kernels can be interpreted as expanding along the depth dimension, so that the nodes 314 of the convolutional layer 304 are arranged also as a (d+1)-dimensional matrix, wherein the size of the (d+1)-dimensional matrix with respect to the depth dimension is by a factor of the number of kernels larger than in the preceding layer 302.

The advantage of using convolutional layers 304 is that spatially local correlation of the input data can exploited by enforcing a local connectivity pattern between nodes of adjacent layers, in particular by each node being connected to only a small region of the nodes of the preceding layer.

In embodiment shown in FIG. 3, the input layer 302 comprises 36 nodes 312, arranged as a two-dimensional 6×6 matrix. The convolutional layer 304 comprises 72 nodes 314, arranged as two two-dimensional 6×6 matrices, each of the two matrices being the result of a convolution of the values of the input layer with a kernel. Equivalently, the nodes 314 of the convolutional layer 304 can be interpreted as arranges as a three-dimensional 6×6×2 matrix, wherein the last dimension is the depth dimension.

A pooling layer 306 can be characterized by the structure and the weights of the incoming edges and the activation function of its nodes 316 forming a pooling operation based on a non-linear pooling function f. For example, in the two dimensional case the values x⁽ⁿ⁾ of the nodes 316 of the pooling layer 306 can be calculated based on the values x⁽ⁿ⁻¹⁾ of the nodes 314 of the preceding layer 304 as

x⁽ⁿ⁾[i,j]=f(x⁽ⁿ⁻¹⁾[id₁,jd₂], . . . ,x⁽ⁿ⁻¹⁾[id₁+d₁−1,jd₂+d₂−1])

In other words, by using a pooling layer 306, the number of nodes 314, 316 can be reduced, by replacing a number d₁·d₂ of neighboring nodes 314 in the preceding layer 304 with a single node 316 being calculated as a function of the values of said number of neighboring nodes in the pooling layer. In particular, the pooling function f can be the max-function, the average or the L2-Norm. In particular, for a pooling layer 306 the weights of the incoming edges are fixed and are not modified by training.

The advantage of using a pooling layer 306 is that the number of nodes 314, 316 and the number of parameters is reduced. This leads to the amount of computation in the network being reduced and to a control of overfitting.

In the embodiment shown in FIG. 3, the pooling layer 306 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 308 can be characterized by the fact that a majority, in particular, all edges between nodes 316 of the previous layer 306 and the nodes 318 of the fully-connected layer 308 are present, and wherein the weight of each of the edges can be adjusted individually.

In this embodiment, the nodes 316 of the preceding layer 306 of the fully-connected layer 308 are displayed both as two-dimensional matrices, and additionally as non-related nodes (indicated as a line of nodes, wherein the number of nodes was reduced for a better presentability). In this embodiment, the number of nodes 318 in the fully connected layer 308 is equal to the number of nodes 316 in the preceding layer 306. Alternatively, the number of nodes 316, 318 can differ.

Furthermore, in this embodiment, the values of the nodes 320 of the output layer 310 are determined by applying the Softmax function onto the values of the nodes 318 of the preceding layer 308. By applying the Softmax function, the sum the values of all nodes 320 of the output layer 310 is 1, and all values of all nodes 320 of the output layer are real numbers between 0 and 1.

A convolutional neural network 300 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 300 can be trained based on the backpropagation algorithm. For preventing overfitting, methods of regularization can be used, e.g. dropout of nodes 312-320, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1. Certain steps or functions of the methods and workflows described herein, including one or more of the steps or functions of FIG. 1, may be performed by a server or by another processor in a network-based cloud-computing system. Certain steps or functions of the methods and workflows described herein, including one or more of the steps of FIG. 1, may be performed by a client computer in a network-based cloud computing system. The steps or functions of the methods and workflows described herein, including one or more of the steps of FIG. 1, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 402 that may be used to implement systems, apparatus, and methods described herein is depicted in FIG. 4. Computer 402 includes a processor 404 operatively coupled to a data storage device 412 and a memory 410. Processor 404 controls the overall operation of computer 402 by executing computer program instructions that define such operations. The computer program instructions may be stored in data storage device 412, or other computer readable medium, and loaded into memory 410 when execution of the computer program instructions is desired. Thus, the method and workflow steps or functions of FIG. 1 can be defined by the computer program instructions stored in memory 410 and/or data storage device 412 and controlled by processor 404 executing the computer program instructions. For example, the computer program instructions can be implemented as computer executable code programmed by one skilled in the art to perform the method and workflow steps or functions of FIG. 1. Accordingly, by executing the computer program instructions, the processor 404 executes the method and workflow steps or functions of FIG. 1. Computer 402 may also include one or more network interfaces 406 for communicating with other devices via a network. Computer 402 may also include one or more input/output devices 408 that enable user interaction with computer 402 (e.g., display, keyboard, mouse, speakers, buttons, etc.).

Processor 404 may include both general and special purpose microprocessors, and may be the sole processor or one of multiple processors of computer 402. Processor 404 may include one or more central processing units (CPUs), for example. Processor 404, data storage device 412, and/or memory 410 may include, be supplemented by, or incorporated in, one or more application-specific integrated circuits (ASICs) and/or one or more field programmable gate arrays (FPGAs).

Data storage device 412 and memory 410 each include a tangible non-transitory computer readable storage medium. Data storage device 412, and memory 410, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 408 may include peripherals, such as a printer, scanner, display screen, etc. For example, input/output devices 408 may include a display device such as a cathode ray tube (CRT) or liquid crystal display (LCD) monitor for displaying information to the user, a keyboard, and a pointing device such as a mouse or a trackball by which the user can provide input to computer 402.

Any or all of the systems and apparatus discussed herein may be implemented using one or more computers such as computer 402.

One skilled in the art will recognize that an implementation of an actual computer or computer system may have other structures and may contain other components as well, and that FIG. 4 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method comprising:
generating a physiological model of an anatomical object of interest of a patient;
receiving electroanatomical mapping data of the anatomical object of interest;
updating the physiological model based on the electroanatomical mapping data of the anatomical object of interest;
performing a simulation for the anatomical object of interest using the updated physiological model; and
outputting results of the simulation.

2. The method of claim 1, wherein the electroanatomical mapping data comprises a 3D map of the anatomical object of interest having electrophysiological data represented at anatomical locations thereon.

3. The method of claim 2, wherein the electrophysiological data comprises at least one of activation time and potentials, voltage, and propagation.

4. The method of claim 1, wherein updating the physiological model based on the electroanatomical mapping data of the anatomical object of interest comprises:
refining conduction velocity values based on activation potential values in the electroanatomical mapping data.

5. The method of claim 1, wherein the electroanatomical mapping data comprises intraoperative data received during a medical procedure, and wherein performing a simulation for the anatomical object of interest using the updated physiological model comprises:
performing a simulation of the medical procedure for the anatomical object of interest using the updated physiological model.

6. The method of claim 1, wherein performing a simulation for the anatomical object of interest using the updated physiological model comprises:
performing a simulation of an ablation procedure on a heart of the patient.

7. The method of claim 1, further comprising:
predicting an optimal therapy using a trained machine learning based model based on information from the updated physiological model and the results of the simulation.

8. The method of claim 1, further comprising:
updating the physiological model based on intraoperative results of an ablation performed during an ablation procedure,
wherein performing a simulation for the anatomical object of interest using the updated physiological model comprises performing a simulation of a next ablation of the ablation procedure based on the updated physiological model.

9. An apparatus comprising:
means for generating a physiological model of an anatomical object of interest of a patient;
means for receiving electroanatomical mapping data of the anatomical object of interest;
means for updating the physiological model based on the electroanatomical mapping data of the anatomical object of interest;
means for performing a simulation for the anatomical object of interest using the updated physiological model; and
means for outputting results of the simulation.

10. The apparatus of claim 9, wherein the electroanatomical mapping data comprises a 3D map of the anatomical object of interest having electrophysiological data represented at anatomical locations thereon.

11. The apparatus of claim 10, wherein the electrophysiological data comprises at least one of activation time and potentials, voltage, and propagation.

12. The apparatus of claim 9, wherein the means for updating the physiological model based on the electroanatomical mapping data of the anatomical object of interest comprises:
means for refining conduction velocity values based on activation potential values in the electroanatomical mapping data.

13. The apparatus of claim 9, wherein the electroanatomical mapping data comprises intraoperative data received during a medical procedure, and wherein the means for performing a simulation for the anatomical object of interest using the updated physiological model comprises:
means for performing a simulation of the medical procedure for the anatomical object of interest using the updated physiological model.

14. The apparatus of claim 9, wherein the means for performing a simulation for the anatomical object of interest using the updated physiological model comprises:
means for performing a simulation of an ablation procedure on a heart of the patient.

15. A non-transitory computer readable medium storing computer program instructions, the computer program instructions when executed by a processor cause the processor to perform operations comprising:
generating a physiological model of an anatomical object of interest of a patient;
receiving electroanatomical mapping data of the anatomical object of interest;
updating the physiological model based on the electroanatomical mapping data of the anatomical object of interest;
performing a simulation for the anatomical object of interest using the updated physiological model; and
outputting results of the simulation.

16. The non-transitory computer readable medium of claim 15, wherein the electroanatomical mapping data comprises a 3D map of the anatomical object of interest having electrophysiological data represented at anatomical locations thereon.

17. The non-transitory computer readable medium of claim 16, wherein the electrophysiological data comprises at least one of activation time and potentials, voltage, and propagation.

18. The non-transitory computer readable medium of claim 15, wherein updating the physiological model based on the electroanatomical mapping data of the anatomical object of interest comprises:
refining conduction velocity values based on activation potential values in the electroanatomical mapping data.

19. The non-transitory computer readable medium of claim 15, the operations further comprising:
predicting an optimal therapy using a trained machine learning based model based on information from the updated physiological model and the results of the simulation.

20. The non-transitory computer readable medium of claim 15, the operations further comprising:
updating the physiological model based on intraoperative results of an ablation performed during an ablation procedure,
wherein performing a simulation for the anatomical object of interest using the updated physiological model comprises performing a simulation of a next ablation of the ablation procedure based on the updated physiological model.
